(19) United States
(12) Patent Application Publication
(10) Pub. No.: US 2004/0003440 A1
(43) Pub. Date: Jan. 1, 2004

(54) Novel L. x formolongo lilies

(76) Inventor: Neil Owen Anderson, St. Paul, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/403,725

(22) Filed: Mar. 31, 2003

Related U.S. Application Data
(60) Provisional application No. 60/369,373, filed on Apr. 1, 2002.

(51) Int. Cl. A01H 5/00

(52) U.S. Cl. 800/321

(57) ABSTRACT

Methods of selecting L.×formolongo lily plants having novel properties are disclosed. The novel properties include day-neutrality, frost-tolerance, reflowering, an increased number of shoots per bulb and an increased number of flowers per shoot.

### CROSS REFERENCE TO RELATED APPLICATIONS

This application claims priority under 35 U.S.C. §119(e) of U.S. Application No. 60/369,373, filed Apr. 1, 2002.

### TECHNICAL FIELD

This invention relates to plant breeding, and more particularly to lily plants with novel properties and flowers produced therefrom.

### BACKGROUND

The genus Lilium (lilies) comprises about 85 species classified into seven sections, including Lilium, Leucolirion, Sinomartagon, Pseudolirium, Martagon, and Archelirion. Five horticultural lily groups are important commercially, i.e. trumpets, Asiatics, Orientals, Aurelian hybrids, and Martagons. Asiatics and Orientals (colored lilies) are predominantly grown as cut flowers, although flowering potted types are also produced. Asiatic hybrids (most commonly derived from the section Sinomartagon, including species such as L. tigrinum, L. bulbiferum, L. dauricum, L. cernuum, L. concolor) have flower colors of orange, red, yellow, white, and tan with the usually non-fragrant flowers facing upwards. Important Asiatic cultivars include ‘Enchantment’, ‘Connecticut King’, ‘Aristo’, ‘Snowstar’, ‘Elite’, ‘Electric’, ‘Bright Beauty’, and ‘Mona’. A new class of Asiatic lilies, the L.A. hybrids, has recently been commercialized. L.A. hybrids resulted from crossing Easter lily (L. longiflorum, section Leucolirion) with Asiatics, producing flowers that are oriented outward (horizontally). Orientals (most commonly derived from species such as L. auratum, L. speciosum, L. rubellum) have red, pink, and white fragrant flowers oriented horizontally. Commercial Oriental cultivars include ‘Star Gazer’, ‘Casa Blanca’, ‘Berlin’, and ‘Marco Polo’. A new class of Oriental lilies, the L.O. hybrids, has been recently commercialized. L.O. hybrids results from a cross of Easter lily with an Oriental lily. Trumpet lily flowers are fragrant and form long tubular shapes that flare open at the ends, forming a trumpet oriented horizontally. Easter lily is the best known trumpet lily, grown commercially as flowering potted plants for sales during the two weeks prior to Easter. See U.S. Pat. No. 4,604,824. Easter lily cultivars are grown in specific countries and include ‘Nellie White’ (U.S.), ‘Snow Queen’ (Europe), ‘Gelria’ (Europe), ‘White American’ (Europe), ‘Osnat’ (Israel, Europe), and ‘Shovav’ (Israel). Martagon types have smaller, non-fragrant flowers that face down vertically with petals reflexed upwards. Most horticultural groups, as well as wild species, are widely cultivated by home gardeners. See Dole and Wilkins, 1999; McRae, 1998.

Lily species and cultivars are also classified into four categories for the time of flower bud initiation and development: (I) flower buds initiate in late summer and continue to develop into late autumn (early flowering Asiatic & Oriental species; ˜31% of Asiatic types are in this category), (II) flower bud initiation occurs in later summer although flower bud development is not completed until the following spring (Asiatic & Oriental species, e.g. L. rubellum), (III) flower bud initiation and development occurs in the spring prior to shoot emergence above the soil (Asiatic & Oriental species), and (IV) flower bud initiation and development occurs in the spring after shoot emergence (Asiatics—later flowering types ˜69%, Easter lily and most other trumpets). See Baranova, 1972. All Lilium species and cultivars require an extended exposure to a cold, moist treatment (vernalization) for rapid stem elongation and emergence above the soil.

Commercial potted and cut lily cultivars in all horticultural classes are 100% vegetatively propagated (bulbs, bulb divisions, aerial axillary stem bulbils), rather than sexually (seed). Garden lily cultivars are predominantly vegetatively propagated, although several seed-propagated cultivars now exist. See Beattie and White, 1993. Potted Easter lilies rank in the top ten flowering potted plants sold in the United States. In the U.S., ‘Nellie White’ is the sole Easter lily cultivar grown, while in Europe, Israel, and Japan other cultivars are produced. See McRae, 1998; Parks, 2002; U.S.D.A., 2001.

Lilies have underground storage organs (bulbs) and are classified as geophytes. Bulbs contain primarily modified leaf tissue (scales). Lily bulbs are imbricated with a configuration of fleshy scales stacked like bricks. A commercial 2-3 year old bulb has inner or daughter scales (from the most recent growing season), outer or mother scales (>1 year old; from the previous growing season), and an apical meristem (growing point) with leaves. The inner scales have not been exposed to a cold period (winter) and contain chemical compounds, inhibitors such as abscisic acid, that prevent stem elongation and shoot emergence without a cold treatment (vernalization). Both the scales and meristems are attached to the basal plate, a compressed stem that constitutes the base of the bulb. Roots emerge from the basal plate. Bulbs function as storage organs for carbohydrates and are never completely dormant, e.g. bulbs that have not initiated flower buds after being dug in the field continue to make leaves. A bulb goes through at least a two- to three-year growth cycle before it is ready to be forced into a flowering, potted Easter lily plant or to produce trumpet, Asiatic, or Oriental cut flowers. During this three-year period, bulbs increase in size, storing nutrients that permit growth of a flowering stem. Bulbs are affected by the climate and growing conditions in each year of growth such that no two sequential crops of a lily clone behave alike with respect to the flowering stem. This variation makes it difficult for a lily forcer to have lily plants or flowers ready in a predictable manner. In addition, since the bulb has gone through a three-year cycle, there is an increased chance of virus infection, root rot, vascular wilt or other diseases incited by plant pathogens. See Dole and Wilkins, 1999.

Easter lilies are commercially propagated in three primary regions: Japan, the United States, and Europe (the Netherlands). In the first year of a commercial bulb propagation cycle (scaling), the bulb propagator/grower starts with the scales of a mother bulb. Outer and inner scales are removed from the basal plate and planted upright in moist soil (peat moss, vermiculite, or sand) with the scale tips above the soil line. The scales form bulblets of one to three inches in circumference with a root system and shoots. In the fall of the year after scaling, the bulblets are dug, sorted, cleaned, and graded. During this process many of the roots and shoots are removed. In the spring of the second year, the bulblets are planted in a field and grown to increase in size. The bulblet is now referred to as a bulb or a yearling. The average size of the bulb at the end of the second year is three to seven inches in circumference, with the larger ones being ready to sell. In the fall, the bulbs are again dug up, cleaned and graded. During this process many of the roots and tops are again removed. In the third year, the bulbs are planted in fields in the spring and continue to enlarge to about seven to ten inches in circumference, at which time they are dug up and ready to go to market. Commercial grades of lily bulbs in the U.S. are ⅞, 8/9, 9/10 inches (circumference). The smaller sized bulbs (⅞) produce plants with fewer flowers than 8/9 or larger sizes and are used for Easter lily sales to mass markets. Larger sized bulbs produced greater flower numbers and command higher prices at independent retailers. Bulbs that are 9/10 frequently consist of two growing points (apical meristems) and produce two flowering stems per bulbs that flower simultaneously, commonly known as double nose bulbs.

In October of the third year, Easter lily bulbs are shipped to a greenhouse grower who forces the bulbs for Easter. The objective is to have the plant fully grown and flowering at the desired time (the Easter holiday date varies from calendar year to year) to provide cut flowers for the flower market or potted plants for the potted plant market.

Commercial forcing of Asiatic lilies for the cut flower market involves an obligate vernalization requirement. Lily bulbs are vernalized for six to ten weeks (cultivar dependent) at 36° to 41° F. under moist conditions prior to greenhouse forcing. Oriental cut lilies require eight to ten weeks of vernalization at 36° to 39° F. prior to greenhouse forcing. See U.S. Pat. No. 5,138,794. Natural cooling also can be used to vernalize by placing the bulbs in the ground. The advantage of natural cooling is that it increases the number of flowers per stem. It is also possible to plant the bulbs in greenhouses and force them to flower by adjusting the temperature and/or the day length. If it is desirable to delay flowering, the bulbs are stored at a high temperature, for example 60° F., for extended periods. Thereafter, the bulbs are precooled and forced. Alternate methods of forcing are known. Commercial forcing of cut Asiatic and Oriental lilies requires continuous vernalization and forcing of sequential bulb lots to ensure year-round production. Each bulb lot is grown and harvested once. See DeHertogh, 1996; U.S. Pat. No. 5,058,318.

For the potted plant market, the same procedures are used as for the cut flower market except that the bulbs are placed in pots either prior to, or immediately after, the vernalization treatment. Easter lilies also have an obligate vernalization requirement of six weeks or 1,000 hours of 40° to 45° F. in moist conditions. Bulbs are vernalized either in the shipping cases (case-cooled), in pots after rooting for one to three weeks at 63° to 65° F. (controlled temperature forcing, CTF), or naturally in pots placed out-of-doors (after rooting for one to three weeks at 63° to 65° F. Case-cooling requires only a limited cooler space for vernalization and can be performed by a supplier or grower. CTF and natural cooling are done only by growers. Easter lilies that have been vernalized by natural cooling have higher leaf and flower numbers than lilies vernalized by case-cooling, as well as longer basal leaves, and shorter plants. If the length of the out-of-doors cold treatment is insufficient, growers must also provide long day photoperiods after shoot emergence above the soil to substitute for the insufficient cold period. CTF provides better vernalization control with uniform conditions each year, plants have high leaf and flower numbers, long basal leaves, but requires more cooler space than case-cooling. See Dole and Wilkins, 1999. Temperatures greater than 70° F. will devernalize treated bulbs, if applied immediately after vernalization. See Miller and Kiplinger, 1966.

Easter lilies have an obligate requirement of a moist, cold treatment (vernalization) for flowering. If three-year bulbs are never vernalized and grown at temperatures greater than 70° F. they will not flower. Vernalization is important for uniform emergence, breakdown of chemical inhibitors found in the inner scales. If insufficient vernalization (less than six weeks at 40° to 45° F.) is applied to either case-cooled, CTF, or natural cooled lily bulbs, long day photoperiods can provide an equal substitution for the lack of cold after shoot emergence. See Lange and Heins, 1988; Roh and Wilkins, 1973; Weiler and Langhans, 1968, 1972. Long day photoperiods consist of twelve or more hours of lighting. Typically, a lily grower will grow emerged, vernalized lilies under eight (0800-1600 HRS) or ten (0700-1700 HRS) hour photoperiods with an additional four-hour night interruption (2200-0200 HRS) using incandescent lights at a minimum of 10 fc or 2 μmols s⁻¹m⁻². See Waters and Wilkins, 1967; Wilkins and Roh, 1970. In the southern-most Japanese-island archipelago, the Ryuku Islands (27° N lat.) where Lilium longiflorum are native, wild Easter lilies flower after being subjected to long days and a short duration of temperatures below 70° F. See Wilkins, 1973. Easter lily bulbs will naturally flower for the home gardener in mid-summer (July) during natural long day photoperiods. Easter lilies will eventually flower if exposed to short day photoperiods after vernalization and shoot emergence. Plants treated with short days would never flower by the sales target date of Easter. Thus, Easter lilies are quantitative long-day plants at the post-shoot emergence stage, which means they will initiate flower buds faster under long days than with short days. Short-day plants require a dark period exceeding some critical length in order to flower, and long-day plants are inhibited from flowering when the dark period exceeds some critical length. Flower bud initiation occurs after shoot emergence and long-day photoperiod treatment during the last two weeks of January (January 28^{th} is the average date), after which flower bud development commences.

As soon as flower bud initiation is complete, the apical meristem ceases making leaves. From this date, a finite number of leaves must be unfolded before the date flower buds are visible (commercially known as visible bud date). An Easter lily crop can be properly timed to flower for Easter by determining the average leaf number for the crop. Typically, three to five plants from each shipment are randomly selected and dissected to estimate the average leaf number, which is divided into the categories: number of unfolded leaves (those leaves at a 45° or greater angle in relation to the perpendicular stem) and number of leaves yet to unfold (those leaves at an angle of less than 45° in relation to the perpendicular stem). The number of unfolded leaves is divided by the number of days from emergence to the present date to calculate the average number of leaves unfolded per day. Next the visible bud date is determined, which is 30 to 35 days prior to the market date (usually Palm Sunday) when the plants are at the puffy bud stage. Twenty-eight days are required from visible bud date to puffy bud stage when plants are grown at 70° F. Then, the number of leaves yet to unfold is divided by the number of days from the day of leaf counting until visible bud date. This is the average number of leaves that must be unfolded each day to reach visible bud date on time. A lily grower may manipulate leaf-unfolding rates by increasing (if behind schedule) or decreasing (if ahead of schedule) the daytime forcing temperatures. See Dole and Wilkins, 1999.

During the extended bulb propagation and production cycle, there is a serious risk of disease (viruses; root rots—Cylindrocarpon radicicola, Fusarium oxysporum, Pythium spp., Rhizoctonia solani, Phytophthora cactorum, P. parasitica) and insect infestation (Rhizoglyphus echinopus—bulb mites) that impair bulb and plant quality. Until World War II and the occurrence of serious disease problems in Japan, the United States imported all Easter lily bulbs from Japan. Commercial cultivars are also susceptible to fluoride (sources are irrigation water, superphosphate fertilizer, perlite, and German peat moss) that causes leaf scorch (leaf tips and leaf margins turn yellow and then brown). Additionally, the lengthy vernalization period and subsequent long-day photoperiod treatments are costly for growers. Lily production for potted plants, cut flowers, or perennial garden types use lily germplasm that does not reflower. Thus, only one harvestable unit is possible in one growing season. Therefore, there is a need for methods that reduce the attendant disadvantages of the present methods for lily production. See Horst, 1990; Wilkins, 1980; Marousky and Woltz, 1977.

### SUMMARY

A method of making a lily plant is disclosed herein. The method comprises the steps of selecting one or more L.×formolongo plants that form flowers under short day photoperiod conditions in a greenhouse environment. The method can further comprise the step of propagating the one or more selected plants. Propagation can comprise asexual propagation or sexual propagation. The one or more selected L.×formolongo plants can be grown from seeds or from bulbs. In some embodiments, the selected L.×formolongo plants are not vernalized. In some embodiments, the one or more selected L.×formolongo plants are progeny of a cross of a L.×formolongo plant and a plant of the parental species, L. longiflorum and L. formosanum. The one or more selected L.×formolongo plants can be progeny of a cross of an (L.×formolongo) plant and a plant of a Lilium species other than L. formosanum and L. longiflorum.

In another aspect, the invention features a method of making a lily plant, comprising the step of selecting one or more L.×formolongo plants that have an average reflowering period of from about 0 to about +176 days under long day photoperiod conditions in a greenhouse environment. The reflowering period of such plants can be from about 0 to about +60 days, e.g., about 0 days to about +7 days, or about +8 days to about +21 days, or from about +22 days to about +60 days. The method can further comprise the step of propagating the one or more selected plants, asexually or sexually. The one or more selected L.×formolongo plants can be grown from seeds or from bulbs. In some embodiments, the selected L.×formolongo plants are not vernalized. In some embodiments, the one or more selected L.×formolongo plants are progeny of a cross of a L.×formolongo plant and a plant of the parental species, L. longiflorum and L. formosanum. The one or more selected L.×formolongo plants can be progeny of a cross of an (L.×formolongo) plant and a plant of a Lilium species other than formosanum or longiflorum.

In another aspect, the invention features a method of making a lily plant that comprises the step of selecting one or more L.×formolongo plants that form 2 to 3 shoots per bulb in a greenhouse environment. The method can further comprise the step of propagating the one or more selected plants. Propagation can comprise asexual propagation or sexual propagation. The one or more selected L.×formolongo plants can be grown from seeds or from bulbs. In some embodiments, the selected L.×formolongo plants are not vernalized. In some embodiments, the one or more selected L.×formolongo plants are progeny of a cross of (L.×formolongo)×L. longiflorum. The one or more selected L.×formolongo plants can be progeny of a cross of an (L.×formolongo) plant and a plant of a Lilium species other than formosanum or longiflorum. The invention also features an L.×formolongo plant that forms 2 to 3 shoots per bulb in a greenhouse environment.

The invention also features a method of making a lily plant, comprising the step of selecting one or more L.×formolongo plants that have a statistically significantly greater number of flowers when grown under short-day conditions in a greenhouse environment relative to the number of flowers when grown under long-day conditions in a greenhouse environment. The one or more progeny plants can be grown from seeds or grown from bulbs. In some embodiments, the one or more progeny plants are not vernalized. The method can further comprise the step of propagating the one or more selected plants, asexually or sexually.

In another aspect, the invention features a method of making a lily plant, comprising the steps of selecting one or more L.×formolongo plants that are capable of surviving two consecutive diurnal periods in which the minimum ambient air temperature is from about—10° C. to about +5° C. In some embodiments, selected plants are capable of surviving more than two consecutive diurnal periods, e.g., ten occurrences, twenty occurrences, thirty occurrences, or forty occurrences. The method can further comprise the step of propagating the one or more selected plants, asexually or sexually.

In another aspect, the invention features a method of making a lily plant, comprising the steps of selecting one or more L.×formolongo plants that are incapable of surviving two consecutive diurnal periods in which the minimum ambient air temperature is from about—10° C. to about +5° C. The method can further comprise the step of propagating the one or more selected plants, asexually or sexually.

In another aspect, the invention features a method of making a lily plant, comprising the steps of selecting one or more L.×formolongo plants that flower in the absence of vernalization. The method can further comprise the step of propagating the one or more selected plants, asexually or sexually.

In another aspect, the invention features a method of making one or more lily bulbs. The method comprises growing a plurality of L.×formolongo plants to form bulbs. Such plants have one or more of the following traits: formation of flowers under short day photoperiod conditions in a greenhouse environment, an average reflowering period of from about 0 to about +176 days under long day photoperiod conditions in a greenhouse environment, formation of 2 to 3 shoots per bulb in a greenhouse environment, a statistically significantly greater number of flowers when grown under short-day conditions in a greenhouse environment relative to the number of flowers when grown under long-day conditions in a greenhouse environment, formation of flowers in the absence of vernalization, survival of two consecutive diurnal periods in which the minimum ambient air temperature is from about −10° C. to about +5° C., and lack of survival of two consecutive diurnal periods in which the minimum ambient air temperature is from about −10° C. to about +5° C.

In another aspect, the invention features a method of making one or more lily flowers. The method comprises growing a plurality of L.×formolongo bulbs to form one or more flowering plants. Such plants have one or more of the following traits: formation of flowers under short day photoperiod conditions in a greenhouse environment, an average reflowering period of from about 0 to about +176 days under long day photoperiod conditions in a greenhouse environment, formation of 2 to 3 shoots per bulb in a greenhouse environment, a statistically significantly greater number of flowers when grown under short-day conditions in a greenhouse environment relative to the number of flowers when grown under long-day conditions in a greenhouse environment, formation of flowers in the absence of vernalization, survival of two consecutive diurnal periods in which the minimum ambient air temperature is from about −10° C. to about +5° C., and lack of survival of two consecutive diurnal periods in which the minimum ambient air temperature is from about −10° C. to about +5° C. In some embodiments, lily flowers are made from bulbs grown under controlled temperature forcing conditions. In other embodiments, lily flowers are made from bulbs grown under case cooling conditions.

In some embodiments of the above methods, a plant of a Lilium species other than L. formosanum or L. longiflorum is a plant selected from the group consisting of an Asiatic lily, an Oriental lily, an L.A. Hybrid lily, an L.O. Hybrid lily, an Aurelian Hybrid lily, a wild lily or a Martagon lily.

The invention also features L.×formolongo seed. Such seed is selected from the group consisting of F₂₊ₙ Lilium formosanum x L. longiflorum seed, and F₁₊ₙBC₁₊ₘ L.×formolongo seed. The invention additionally features seed of a cross of a (L.×formolongo) plant and a plant of a Lilium species other than L. formosanum and L. longiflorum. Species other than L. formosanum and L. longiflorum include Asiatic lily, Oriental lily, L.A. Hybrid lily, L.O. Hybrid lily, Aurelian Hybrid lily, and Martagon lily. Plants grown from the foregoing seed exhibit a trait selected from the group consisting of formation of flowers under short day photoperiod conditions in a greenhouse environment, an average reflowering period of from about 0 to about +176 days under long day photoperiod conditions in a greenhouse environment, formation of 2 to 3 shoots per bulb in a greenhouse environment, a statistically significantly greater number of flowers when grown under short-day conditions in a greenhouse environment relative to the number of flowers when grown under long-day conditions in a greenhouse environment, formation of flowers in the absence of vernalization, survival of two consecutive diurnal periods in which the minimum ambient air temperature is from about −10° C. to about +5° C., and lack of survival of two consecutive diurnal periods in which the minimum ambient air temperature is from about −10° C. to about +5° C.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DETAILED DESCRIPTION

It has been discovered that progeny of a cross of two trumpet lilies, L. formosanum x L. longiflorum, show segregation for a number of useful traits, including day neutrality, facultative or non-obligate vernalization requirement, reflowering, frost-tolerance, multiple shoots per bulb and multiple flowers per plant. These hybrids are commonly known as L.×formolongo or L.×formolongi. These properties are useful for cut and potted lily growers (propagators, pre-finishers, and finishers), as well as home gardeners.

L.×formolongo Plants

L.×formolongo plants for use in the invention are made from a cross of Lilium formosanum and Lilium longiflorum, using known techniques. Both species are white trumpet lilies with the former native to Formosa and the latter native to the Ryuku Islands. In most instances, L. formosanum is the female parent in the cross. L. formosanum typically serves as the female parent because of unilateral incompatibility favoring pollen tube growth in this crossing direction (Ascher and Peloquin, 1968). Known L.×formolongi hybrids flower within one year from seed and have the characteristic of wide leaves as does L. longiflorum (Okazaki, 1996). L. longiflorum and L. formosanum are closely related species in the section Leucolirion. They differ cytologically in that L. longiflorum has three additional intercalary C-bands on chromosomes g, i, and l compared to L. formosanum (Smyth et al., 1989).

The F₁ descendants of a cross of Lilium formosanum and Lilium longiflorum can be used in methods of the invention. Alternatively, F₂, F₃, F₄, etc. descendants can be used in methods of the invention, also referred to herein as F₂₊ₙdescendants. In other embodiments, F₁, F₂, F₃, i.e., F₁₊ₙ descendants, can be backcrossed to a plant of one of the parent species, L. longiflorum and L. formosanum, one or more times to create, for example, F₁₊ₙBC₁₊ₘ progeny, e.g., F₁BC₁ progeny, or F₁BC₂ progeny. Typically, n is an integer from 0 to 10 and m is an integer from 0 to 10. Such progeny can also be used in methods of the invention. F₁ L.×formolongo plants, F₁₊ₙBC₁₊ₘ L.×formolongo plants, and F₂₊ₙ L.×formolongo plants can be grown using known techniques from suitable plant parts, e.g., seeds, bulbs, and leaf cuttings.

In some embodiments, a breeder can cross L.×formolongo plants, either as males or females, with wild species, colored Asiatic, Oriental, L.A. Hybrid, L.O. Hybrid, Aurelian Hybrid, or Martagon commercial horticultural groups of lilies. The progeny of such crosses can then be subjected to selection as described herein, recognizing that the frequency of individuals exhibiting the selected trait will be lower in such progeny.

Typically, a practitioner carries out one or more pollinations to create a population of L.×formolongo plants. In some embodiments, however, a practitioner obtains a population of L.×formolongo plants from another source. The number of plants to be used in a method depends upon, inter alia, the space available for growing plants and the likelihood of obtaining a segregant that exhibits one or more of the properties described below. Thus, for example, from about 100 to about 1,000 plants, or about 10 to about 50 plants can be prepared or obtained, and subjected to selection for the desired trait. However, it should be appreciated that as few as one L.×formolongo plant or progeny thereof can be used in the methods.

Day Neutrality

Non-vernalized L. longiflorum, L. formosanum lily plants do not develop flowers under either long or short-day conditions. Vernalized L. longiflorum will eventually flower under short days, a significantly greater number of days to flower than under long days. It has now been discovered that non-vernalized L.×formolongo progeny can be selected that develop flowers under either short day and/or long day photoperiods.

Thus, in one aspect, the invention features a method of making L.×formolongo hybrid lily plants that initiate and develop flower buds under short and long day photoperiod conditions (without a vernalization treatment) in a greenhouse environment. Lily plants that can form flowers under such conditions are also referred to herein as day neutral lilies.

L.×formolongo plants are grown in a greenhouse environment under short day and long day photoperiod conditions. Greenhouse conditions include a temperature of from about 60° F. to about 75° F. (24 hours per day) using natural lighting. In L. longiflorum, one of the parents of L.×formolongo, a long day photoperiod (eight hours of light, 0800-1600 HRS plus night interruption lighting, 2200-0200 HRS) is not perceived by the shoot tip at temperatures over 70° F./21° C. See Lange and Heins, 1988; Roh and Wilkins, 1973. However, if greenhouse temperatures of L. longiflorum are at or below 70° F./21° C. for any period of time, photoperiod may elicit a flowering response. Thus, both short and long day photoperiods are necessary in the selection process of L.×formolongo. Supplemental lighting with high intensity discharge lamps (50-150 μmols s⁻¹m⁻²) is often used in greenhouses during winter when light conditions are low (e.g., in northern latitudes). Short day photoperiod conditions include light at 0800-1600 HRS; long day photoperiods also have eight hours of light during the day 0800-1600 HRS plus night interruption lighting, 2200-0200 HRS. Night interruption lighting is supplied either by incandescent or high intensity discharge lights at ≧10 fc or 2 μmols s⁻¹m⁻² (at plant height).

Selection can be carried out by visual inspection of plants (seedlings) for the presence of one or more visible buds, e.g., inspection on a daily or weekly basis after flower bud initiation has occurred and flower bud development has progressed to visible bud date. Alternatively, selection can be carried out by visual inspection of plants for the presence of one or more developed inflorescences. Although the segregation frequency of the day neutrality trait in L.×formolongo progeny may vary, it is contemplated that about 20% up to about 40% of the L.×formolongo F₁ or F₁BC₁ progeny will form flowers. Typically, if no progeny have produced flowers after 400 days under short and/or long day photoperiod conditions in a greenhouse environment, it is likely that no segregants will be identified from that particular progeny population.

The selected L.×formolongo plants can be propagated. Propagation can be asexual, for example, from bulbs, from leaf cuttings, from stem cuttings, from tissue culture, or from scales. Asexual propagation is useful when it is desired to maintain and expand the selected genotype to produce large numbers of bulbs for commercial production. In some embodiments, propagation can be sexual, e.g., when it is desired to use the selected genotype in a breeding program or to create a seed-propagated hybrid or inbred cultivar.

In some embodiments, L.×formolongo plants are not vernalized prior to placing them in the greenhouse under short day and long day photoperiods. Plants that flower without vernalization are useful because they require less manipulation and do not require a low temperature incubation period. That L.×formolongo plants do not require vernalization is significant, since vegetatively-propagated L. longiflorum parental clones have an obligate requirement for vernalization for stem elongation prior to flower bud initiation and development.

L.×formolongo lily plants that exhibit day neutrality are useful because commercial crop plants can be grown under a short day photoperiod (without a vernalization pre-treatment), in the absence of additional exposure to non-natural light (night interruption lighting) in order to flower. Thus, growers need not implement long day photoperiods after shoot emergence in order to compensate for inadequate vernalization. Therefore, lily crops can be grown throughout the year, even in those seasons with natural short day conditions. Likewise, commercial crops can be grown under a long day photoperiod (without a vernalization pre-treatment) in order to flower, permitting flower production under natural long day conditions. During periods of low natural irradiance, supplemental lighting can be provided to the crop for improved growth without concern for long days substituting for vernalization.

Reflowering

In another aspect, the invention features a method of making a lily plant that has an average reflowering period of a desired duration. L.×formolongo progeny plants for use in the method are prepared or obtained as described above.

Selection is carried out on the L.×formolongo population by growing the plants under short day and long day photoperiod conditions in a greenhouse environment, and identifying segregants that exhibit a reflowering period of the desired duration. Long day and short day photoperiods and suitable greenhouse conditions are described above. In some embodiments, the L.×formolongo plants are not vernalized prior to placing them in the greenhouse under long day photoperiod conditions.

Reflowering period is calculated as the average number of days between the date the first flower opens on the initial terminal shoot and the date the first flower opens on the second shoot. The reflowering period within a seedling population is recorded for each individual plant (genotype with unique or similar genetic constitution to other members of the seedling's population) and a population average can be derived. To determine the average reflowering period of an individual seedling, it is useful to asexually propagate each individual seedling of the population, in order to have a plurality of plants with which to calculate the average.

Selection can be applied to obtain L.×formolongo plants that have an average reflowering period from about +0 to about +176 days under short day and/or long day photoperiod conditions in a greenhouse environment, e.g., from about 0 to about +7 days, from about +2 to about +7 days, from about +8 days to about +21 days, or about +22 days to about +60 days, or about +61 days to about +120 days, or about +121 days to about +176 days. The frequency of L.×formolongo plants in an F₁BC₁ population that have an average reflowering period of from about 0 to about +5 days is contemplated to be about 1% to about 80% under short day photoperiods and about 25% to about 100% under long day photoperiods.

After the second shoot flowers, the next shoot (the third flower stem) will flower, which is followed by the fourth, and so on. Thus, reflowering L.×formolongo selections differ from commercial double nose bulbs (9/10 circumference Easter lily bulbs) described above, which have two simultaneous flowering shoots but do not have the potential for additional shoots to flower in the same growing season.

The selected L.×formolongo plants can be sexually or asexually propagated as described above. Such plants are useful because they can be selected for the number of flowers or the rate of flower production. Selected plants can be useful to growers because such plants generally produce more flowers during the lifetime of the plant or during a growing season than commercially cultivated cultivars of L. longiflorum, L. formosanum. Cut lilies could be grown in greenhouses with year-round flowering production potential or in fields, out-of-doors, with continuous cut flower production during the growing season. In addition, potted lilies could be grown with multiple flowering stems per pot and the potential to reflower for the consumer after purchase thereby increasing the market value of such a product. In the home garden setting, multiple flowering stems of lilies could be produced simultaneously or sequentially, providing flowering lilies throughout the growing season.

In another aspect, a method of making one or more lily flowers is provided by the invention. This method includes growing a plurality of L.×formolongo bulbs of a clone to form one or more flowering plants. Plants are selected that have an average reflowering period in a range from about 0 to about +176 days under short day and/or long day photoperiod conditions in a greenhouse environment. The bulbs can be grown without vernalization or with vernalization under controlled temperature forcing conditions, natural cooling, or under case cooling conditions. Controlled temperature forcing, natural cooling, and case cooling conditions are as described above.

Asexually propagated bulbs that will grow into plants with a reflowering period in a range from about 0 to +176 days under short day and/or long day greenhouse conditions are useful to finishers because, as described above, such plants generally produce more flowers during a growing season or the lifetime of the plant than commercial lilies that do not reflower without an additional vernalization treatment.

Shoots per Bulb

In another aspect, the invention features a method of making a L.×formolongo plant that forms multiple shoots per bulb under greenhouse conditions. It is contemplated that each bulb from such plants can produce, for example, up to 10 shoots during a single growing season (e.g., 2, 3, 4, 5, 6, 7, 8, 9, or 10 shoots). Subsequent shoots on a bulb typically develop from about the time the terminal (first) stem initiates flower buds to about the time of early to completed flower bud development. Therefore, selection for plants that form multiple shoots per bulb is usually performed at the stage from the date the first stem initiates and develops flower buds to the date the tenth stem emerges. The frequency of plants that form multiple shoots per bulb in an F₁BC₁ population is contemplated to be 20% to 80%, although the frequency will vary depending upon the methods and parents used to generate the L.×formolongo plants.

Plants selected for the trait of formation of multiple shoots per bulb can be propagated asexually or sexually, as discussed herein. In some embodiments, the L.×formolongo plants are not vernalized.

An L.×formolongo plant that forms multiple (e.g., 2 to 10) shoots per bulb in a greenhouse environment is useful to growers and finishers because more flowers and/or flower stems are produced per plant. This is particularly advantageous to the cut flower industry where cut flowers could be in continuous production, rather than the current method of harvesting the first and only flower stem and replanting with the next production cycle of vernalized bulbs.

Number of Flowers per Lily Plant

In another aspect, the invention features a method of making a lily plant that produces statistically significantly more flowers when grown in a greenhouse environment under short day conditions relative to the number of flowers produced in a greenhouse environment under long day conditions. Such a method includes providing a plurality of progeny plants of a cross between Lilium formosanum and L. longiflorum and identifying one or more of said progeny plants that have a statistically significantly greater number of flowers when grown under short day conditions in a greenhouse environment relative to the number of flowers when grown under long day conditions in a greenhouse environment.

Conditions in a greenhouse environment with respect to lily plants are discussed above. Photoperiods corresponding to short day conditions and long day conditions are described above. As used herein, “statistically significant” refers to a p-value of less than 0.05, e.g., a p-value of less than 0.025 or a p-value of less than 0.01, using an appropriate measure of statistical significance. For example, non-parametric statistical tests (e.g., Kruskal-Wallis or Chi-square tests) can be used to evaluate plants grown in a greenhouse environment versus plants grown in a growth chamber environment. The same non-parametric statistical tests can be used to evaluate plants grown under short day conditions versus plants grown under long day conditions.

The plants obtained by the method can be propagated by asexual propagation, or by sexual propagation, as discussed herein. In some embodiments, the L. formosanum x L. longiflorum progeny plants do not require vernalization.

Frost-Tolerance and Frost-Sensitivity

In another aspect, the invention features a method of making an L.×formolongo plant that is capable of surviving two consecutive diurnal periods in which the minimum ambient air temperature is from about −10° C. to about +5° C., i.e., a frost period. Such plants are sometimes referred to herein as frost-tolerant plants. Frost-tolerant plants obtained after selection can be propagated by asexual propagation or by sexual propagation, as discussed herein. Selection can be applied to identify L.×formolongo plants that can survive one or more occurrences of a frost period, e.g., 20 occurrences, 30 occurrences, 40 occurrences, 50 occurrences, 60 occurrences, 70 occurrences, 80 occurrences, or 90 occurrences of a frost period. The segregation frequency for survival of 90 occurrences in an L.×formolongo population is estimated to be from about 31.25% to about 62.5%.

Methods of obtaining L.×formolongo progeny for use in the invention are discussed herein. Frost damage can be measured by the amount of stem breakage, “wilting” after sunrise, the presence of water-soaked (dead) regions within a leaf, flower, or stem of said plant. Plants that survive two consecutive diurnal periods typically exhibit a decrease or complete absence of one or more of the following characteristics: stem breakage, “wilting” after sunrise, or water-soaked (dead) regions within a leaf, flower, or stem, compared to the corresponding characteristics exhibited by non-frost-tolerant plants.

Frost-tolerant lily plants are useful because they will tolerate lower growing temperatures which allows these plants to be grown in cool greenhouses or out-of-doors in more Northern climates. These plants are useful to the cut and potted lily plant industry as well as the home lily gardener.

Alternately, L.×formolongo plants can be selected that are incapable of surviving two consecutive diurnal periods in which the minimum ambient air temperature is from about −10° C. to about +5° C. Such plants are referred to as frost-sensitive. Although frost-sensitive plants are less useful for commercial field production, they can have value for growers of greenhouse potted and cut lilies.

Vernalization

In another aspect, the invention features a method of making a lily plant that comprises selecting one or more L.×formolongo plants that flower in the absence of vernalization. Plants obtained following such selection are propagated, either asexually or sexually. L.×formolongo plants that are not vernalized can be grown in a greenhouse environment, and can be grown under short day or long day conditions. The terminal shoot (grown from seedlings or vegetative bulbs) that does not require vernalization typically begins elongation within 90 days to 150 days and flower at 220 days to 306 days after sowing (seeds) or planting (bulbs). Plants that require vernalization will remain rosetted during this period and thereafter. Therefore, selection for plants that do not require vernalization can be performed following about 90 to about 150 days. Approximately 20% to 100% of plants will flower without vernalization, although this number will vary depending upon the method used to generate the L.×formolongo plants and the conditions under which they are grown.

L.×formolongo plants, selected for one or more of the traits described herein, can be grown to form bulbs. In another aspect, L.×formolongo bulbs, selected for one or more of the traits described herein, can be grown to making one or more lily flowers. Such bulbs can be grown under controlled temperature forcing conditions or under case cooling conditions.

The invention further provides for hybrid L.×formolongo seed. Plants grown from such seed possess one or more of the traits described herein. L.×formolongo seed includes F₂₊ₙ Lilium formosanum x L. longiflorum seed, F₁₊ₙBCₙ L.×formolongo seed, or seed from a cross of a (L.×formolongo) plant and a plant of a Lilium species other than formosanum and longiflorum.

L.×formolongo plants that have been selected according to the invention can be crossed either as males or females with wild species, colored Asiatic, Oriental, L.A. Hybrid, L.O. Hybrid, Aurelian Hybrid, or Martagon commercial horticultural groups of lilies. The progeny of such crosses can be subjected to further selection for other desired traits, in order to develop additional varieties for commercial use.

The invention will be further described in the following examples, which do not limit the scope of the invention described in the claims.

### EXAMPLES

### Example 1

### Interspecific Hybrid Lily Plants

Seeds of F₁ L.×formolongo cultivars or varieties ‘Raizan No. 1’ (Seed Lot Nos. 8241; 8159), ‘Raizan No. 2’ (Seed Lot Nos. 8086; 8148), and ‘Sakigake Raizan’ (‘Raizan Herald’; Seed Lot Nos. 2029; 9097) were planted and grown in a greenhouse. Seeds of these varieties were obtained from Dai-Ichii Seed Co., Ltd. (Tokyo, Japan). Bulbs of L. longiflorum variety ‘Nellie White’ were obtained from the Fred Gloeckner Co. (Harrison, N.Y., U.S.A.). Seeds of L. longiflorum variety ‘Snow Trumpet’ (Seed Lot No. 1182) were obtained from Sakata Seed Corp. (Yokohama, Japan). Seeds of wild L. formosanum (open-pollinated, half-sib family) were obtained from Mary Queitzsch (Madison Mills, Va., U.S.A.). Bulbs of wild L. formosanum were purchased from Heronswood Nursery (Kingston, Wash., U.S.A.). The propagation method and vernalization treatment for each variety are shown in Table 1.

TABLE 1

| Species | Cultivar/Variety | Propagation Method | Comments |
|---|---|---|---|
| L. longiflorum | Snow Trumpet | Seed | Non-cooled |
| L. longiflorum | Nellie White | Vegetative | Case cooled |
| | | (9/10 Bulbs) | |
| L. longiflorum | Nellie White | Vegetative | Non-cooled |
| | | (9/10 Bulbs) | |
| L. formosanum | — | Seeds | Non-cooled |
| L. formosanum | — | Vegetative (5/6 Bulbs) | Cooled |
| L. xformolongo | Raizan No. 1 | Seed | Non-cooled |
| L. xformolongo | Raizan No. 2 | Seed | Non-cooled |
| L. xformolongo | Sakigake Raizan | Seed | Non-cooled |

Interspecific hybrids were made by crossing L. formosanum with L. longiflorum accessions. F₁ progeny of these crosses were used as parents in the crosses shown in Table 2. F₁BC₁ progeny (containing two species dosages of L. longiflorum and one species dosage of L. formosanum) of the crosses shown in Table 2 were given the indicated designations and used in the experiments described below.

TABLE 2

| Hybrid | Female Parent | Male Parent |
|---|---|---|
| 00L-13 | (L. formosanum x L. longiflorum) A-2 | ‘Nellie White’ |
| 00L-74 | (L. formosanum x L. longiflorum) R-27 | ‘Nellie White’ |
| 00L-96 | (L. formosanum x L. longiflorum) R-34 | ‘Nellie White’ |
| 00L-116 | (L. formosanum x L. longiflorum) R-6 | ‘Nellie White’ |
| 00L-136 | ‘Nellie White’ | (L. formosanum x |
| | | L. longiflorum) R-1 |

### Example 2

### Segregation of Phenotypes in Hybrid Lily Plants under Greenhouse Conditions

The seed-propagated hybrids, F₁BC₁, and varieties described in Example 1 were germinated and grown for ten weeks, at which time they were transplanted and moved to a greenhouse. Lily bulbs were vernalized at 40° to 45° F. in moist conditions for six weeks (1,000 hours) starting in October. On about the same day that the vernalization treatment began for bulbs, seeds were sown for germination. Photoperiod treatments commenced in December and plants were grown in a greenhouse in Minnesota for about seven months, under conditions essentially as described above. Plants were fertilized weekly with 300 ppm N, using a commercial dark-weather fertilizer (20-20-10 analysis). In between fertilizations, plants were watered with tap water. Greenhouse temperature during the experiment were set at about 70° F./21° C. (constant 24 hours). Temperature recorders were placed at a level approximately equal to the top of the pot, and actual temperature readings were recorded on thermographs for the duration of the experiment. Mean±S.D. temperatures recorded for the short day photoperiod were 79.3±7.9° F. (day) and 64.6±3.8° F. (night). Mean±S.D. temperatures recorded for the long day photoperiod were 80.6±7.9° F. (day) and 66.1±3.6° F. (night). One half of the plants were grown under short day photoperiod conditions of 8 hours day and 16 hours night. The remaining plants were grown under long day photoperiod conditions of 8 hours day followed by 6 hours night (dark) plus 4 hours of night interrupted lighting (day) and then 6 hours night (dark).

Plants were observed weekly until inflorescences were about to appear, at which time plants were observed daily. The mean number of days to visible bud date (VBD), and number of days to first flower were recorded. Measurements were made of plant height from the soil surface (cm), inflorescence length (cm), number of leaves, number of flowers, and number of shoots/bulb. There were five replications per accession (seedlings or bulbs) in each photoperiod. Plants were placed in each photoperiod in a completely randomized design. Tables 3 and 4 show the results for Lilium longiflorum, L. formosanum, and L.×formolongo interspecific F₁ hybrids grown in a greenhouse under short (SD) and long day (LD) photoperiods.

The results show that, as expected, ‘Nellie White’ did not flower during the experiment in the absence of a vernalization treatment. ‘Nellie White’ bulbs that had been case cooled did, as expected, flower earlier under long day conditions than under short day conditions. In contrast, some L.×formolongo hybrid seedlings actually flowered at the same time or even earlier under short day conditions. See, e.g., ‘Raizan No. 1’, Lot 8159 and hybrid 00L-13 in Table 3. With the exception of ‘Raizan No. 1’, Lot 8241 and ‘Raizan No. 2’ Lot No. 8086, the day neutrality trait was segregating in the progeny because 0% to 100% flowering was noted.

TABLE 3

| | Days to | | | | Days to First | | | |
|---|---|---|---|---|---|---|---|---|
| | VBD^{a} | | % Flowering | | Flower | | Plant Height | |
| Accession | SD^{b} | LD^{b} | SD | LD | SD | LD | SD | LD |
| ‘Nellie White’, Case cooled | 157 | 135 | 100 | 100 | 180 | 167 | 29 | 33 |
| ‘Nellie White’, Non-cooled | — | — | 0 | 0 | — | — | 29 | 62 |
| ‘Snow Trumpet’ | 206 | 214 | 100 | 40 | 238 | 231 | 76 | 34 |
| L. formosanum bulbs, case cooled | 233 | 240 | 100 | 100 | 260 | — | 68 | 53 |
| L. formosanum seedlings | 258 | 222 | 60 | 80 | 262 | 250 | 68 | .83 |
| ‘Raizan No. 1’, Lot 8241 | 211 | 196 | 100 | 100 | 244 | 227 | 101 | 98 |
| ‘Raizan No. 1’, Lot 8159 | 195 | 200 | 60 | 0 | 222 | 223 | 93 | 99 |
| ‘Raizan No. 2’, Lot 8086 | 228 | 200 | 100 | 100 | 254 | 229 | 100 | 100 |
| ‘Raizan No. 2’, Lot 8148 | 196 | 191 | 20 | 40 | 226 | 220 | 100 | 109 |
| ‘Sakigake Raizan’, Lot 2029 | 220 | 199 | 80 | 60 | 244 | 228 | 88 | 82 |
| ‘Sakigake Raizan’, Lot 9097 | 201 | 209 | 20 | 20 | 230 | 240 | 107 | 109 |
| Hybrid 00L-13 | 220 | 220 | 80 | 60 | 253 | 238 | 68 | 47 |
| Hybrid 00L-74 | 222 | 215 | 80 | 100 | 250 | 237 | 56 | 74 |
| Hybrid 00L-96 | 256 | 218 | 40 | 100 | 266 | 243 | 57 | 72 |
| Hybrid 00L-116 | 214 | 195 | 80 | 100 | 240 | 223 | 71 | 73 |
| Hybrid 00L-136 | 274 | 203 | 0 | 40 | 277 | 233 | 32 | 40 |

^{a}VBD, visible bud date;
^{b}SD, short day; LD, long day.

TABLE 4

| | Inflorescence | | | | No. of | | No. | |
|---|---|---|---|---|---|---|---|---|
| | Length | | No. of leaves | | Flowers | | of Shoots | |
| Accession, Environment | SD^{a} | LD^{a} | SD | LD | SD | LD | per Bulb | |
| ‘Nellie White”, Case cooled | 17 | 16 | 111 | 98 | 5.6 | 4.4 | 1.0 | 1.0 |
| ‘Nellie White’, Non-cooled | — | — | 171 | 192 | — | — | 1.0 | 1.0 |
| “Snow Trumpet” | 14 | 13 | 45 | 22 | 1.2 | 1.3 | 1.4 | 1.0 |
| L. formosanum bulbs, case cooled | 9 | 12 | 88 | 67 | 1.0 | 1.0 | 1.0 | 1.0 |
| L. formosanum seedlings | 8 | 9 | 73 | 41 | 2.0 | 1.4 | 1.2 | 1.0 |
| ‘Raizan No. 1’, Lot 8241 | 10 | 10 | 63 | 59 | 1.8 | 2.6 | 1.6 | 1.6 |
| ‘Raizan No. 1’, Lot 8159 | 14 | 15 | 60 | 57 | 2.2 | 2.6 | 1.6 | 1.0 |
| ‘Raizan No. 2’, Lot 8086 | 6 | 14 | 60 | 61 | 2.2 | 1.8 | 1.6 | 1.6 |
| ‘Raizan No. 2’, Lot 8148 | 14 | 16 | 60 | 62 | 2.6 | 2.0 | 1.2 | 1.0 |
| ‘Sakigake Raizan’, Lot 2029 | 11 | 11 | 59 | 45 | 1.8 | 2.0 | 2.5 | 1.0 |
| ‘Sakigake Raizan’, Lot 9097 | 16 | 13 | 57 | 65 | 2.4 | 2.4 | 1.0 | 1.4 |
| Hybrid 00L-13 | 12 | 15 | 45 | 30 | 1.0 | 1.2 | 1.2 | 1.2 |
| Hybrid 00L-74 | 13 | 11 | 42 | 36 | 1.0 | 1.0 | 1.8 | 1.8 |
| Hybrid 00L-96 | 7 | 10 | 51 | 40 | 2.2 | 1.0 | 1.2 | 1.4 |
| Hybrid 00L-116 | 14 | 16 | 51 | 45 | 1.4 | 2.4 | 1.8 | 1.2 |
| Hybrid 00L-136 | — | 12 | 36 | 33 | 1.0 | 1.0 | 1.0 | 1.2 |

^{a}SD, short day; LD, long day.

### Example 3

### Segregation of Phenotypes in F₁ Lily Plants under Growth Chamber Conditions

The hybrids and varieties described in Example 1 were also grown from seed under growth chamber conditions. Photoperiod treatments commenced in December and continued for seven months. Typical conditions for growing lilies in a growth chamber were used, essentially as described for greenhouse conditions except that there was no natural lighting (sunlight) during the day and that supplemental lighting was supplied by a combination of incandescent and fluorescent lamps (rather than high intensity discharge lamps). Plants were fertilized weekly with 300 ppm N, using a commercial dark-weather fertilizer (20-20-10 analysis). In between fertilizations, plants were watered with tap water. Growth chamber temperatures were kept at 21° C. during the experiment at a relative humidity of 45-55%. Actual temperatures readings were recorded on thermographs for the duration of the experiment. Mean±S.D. temperatures recorded for the short day photoperiod were 70.3±0.2° F. (day/night). Mean±S.D. temperatures recorded for the long day photoperiod were 70.6±1.9° F. (day/night). One half of the plants were grown under short day photoperiod conditions of 8 hours day and 16 hours night. The remaining plants were grown under long day photoperiod conditions of 8 hours day followed by 6 hours night (dark) plus 4 hours of night interrupted lighting (day) and then 6 hours night (dark).

Measurements were made of mean number of days to visible bud date (VBD), number of days to first flower, plant height (cm), inflorescence length (cm), number of leaves, number of flowers, and number of shoots/bulb. Measurements were made on five plants of each hybrid or variety. Tables 5 and 6 show the results for plants grown under short day and long day photoperiods.

The results in Table 5 and 6 show that day neutrality was exhibited by about 20% of the ‘Raizan No. 1’ plants (Seed Lots 8241, 8159), about 20% of the ‘Raizan No. 2’ plants (Seed Lot No. 8148 only), about 20% of the ‘Sakigake Raizan’ plants (Seed Lots 2029, 9097). About 50% of the F₁BC₁ backcross progeny would have eventually flowered, since about 50% had elongating stems with initiated flower buds at the termination of the experiment.

TABLE 5

| | Days to | | | | Days to First | | | |
|---|---|---|---|---|---|---|---|---|
| | VBD^{a} | | % Flowering | | Flower | | Plant Height | |
| Accession | SD^{b} | LD^{b} | SD | LD | SD | LD | SD | LD |
| ‘Nellie White”, Case cooled | — | 158 | — | 40 | — | 187 | 12 | 22 |
| ‘Nellie White’, Non-cooled | — | — | — | — | — | — | 8 | 18 |
| ‘Snow Trumpet’ | — | — | — | — | — | — | 10 | 8 |
| L. formosanum bulbs, case cooled | 124 | 122 | 100 | 0 | 143 | — | 15 | 29 |
| L. formosanum seedlings | — | — | 0 | 0 | — | — | 10 | 8 |
| ‘Raizan No. 1’, Lot 8241 | 252 | 175 | 20 | 80 | — | 208 | 29 | 30 |
| ‘Raizan No. 1’, Lot 8159 | 240 | 152 | 60 | 0 | 257 | — | 41 | 21 |
| ‘Raizan No. 2’, Lot 8086 | 194 | 27 | 0 | 40 | — | 217 | 22 | 27 |
| ‘Raizan No. 2’, Lot 8148 | 228 | 161 | 20 | 40 | 262 | 204 | 49 | 19 |
| ‘Sakigake Raizan’, Lot 2029 | 234 | 172 | 20 | 20 | 259 | 188 | 41 | 18 |
| ‘Sakigake Raizan’, Lot 9097 | 234 | 212 | 20 | 20 | 259 | 237 | 44 | 38 |
| Hybrid 00L-13 | — | — | 0 | 0 | — | — | 8 | 9 |
| Hybrid 00L-74 | — | — | 0 | 0 | — | — | 7 | 8 |
| Hybrid 00L-96 | — | — | 0 | 0 | — | — | 10 | 7 |
| Hybrid 00L-116 | — | — | 0 | 0 | — | — | 9 | 8 |
| Hybrid 00L-136 | — | — | 0 | 0 | — | — | 9 | 6 |

— = No buds or flowers were observed;
^{a}VBD, visible bud date;
^{b}SD, short day; LD, long day.

TABLE 6

| | Inflorescence | | No. of | | No. of | | No. of | |
|---|---|---|---|---|---|---|---|---|
| | Length | | Leaves | | Flowers | | Shoots | |
| Accession, Environment | SD^{a} | LD^{a} | SD | LD | SD | LD | per Bulb | |
| ‘Nellie White”, Case cooled | — | 7 | 61 | 106 | — | 3.0 | 1.0 | 1.0 |
| ‘Nellie White’, Non-cooled | — | — | 56 | 119 | — | — | 1.0 | 1.0 |
| “Snow Trumpet’ | — | — | 14 | 13 | — | — | 1.0 | 1.0 |
| L. formosanum bulbs, case cooled | 3 | 16 | 146 | 91 | 1.0 | 1.0 | 1.0 | 1.0 |
| L. formosanum seedlings | — | — | 14 | 13 | — | — | 1.0 | 1.0 |
| ‘Raizan No. 1’, Lot 8241 | 8 | 8 | 60 | 47 | 1.0 | 1.25 | 1.5 | 2.0 |
| ‘Raizan No. 1’, Lot 8159 | 6 | 1 | 34 | 41 | 1.0 | 1.5 | 2.0 | 1.6 |
| ‘Raizan No. 2’, Lot 8086 | — | 6 | 41 | 52 | — | 1.0 | 1.0 | 1.3 |
| ‘Raizan No. 2’, Lot 8148 | 8 | 3 | 59 | 36 | 1.0 | 1.0 | 1.8 | 1.5 |
| ‘Sakigake Raizan’, Lot 2029 | 6 | 6 | 30 | 39 | 1.0 | 1.5 | 1.3 | 2.0 |
| ‘Sakigake Raizan’, Lot 9097 | 3 | 6 | 65 | 36 | 1.0 | 2.0 | 1.0 | 2.5 |
| Hybrid 00L-13 | — | — | 18 | 15 | — | — | 1.0 | 1.0 |
| Hybrid 00L-74 | — | — | 11 | 9 | — | — | 1.0 | 1.0 |
| Hybrid 00L-96 | — | — | 20 | 17 | — | — | 1.0 | 1.0 |
| Hybrid 00L-116 | — | — | 15 | 14 | — | — | 1.0 | 1.0 |
| Hybrid 00L-136 | — | — | 13 | 13 | — | — | 1.0 | 1.0 |

^{a}SD, short day; LD, long day.

### Example 4

### Flowering of Interspecific Hybrid Lily Plants under Greenhouse Conditions

The plants of Example 2 were maintained in the greenhouse under the same experimental conditions, after the termination of the seven-month experiment. Plants were observed on a daily basis for the presence of second shoots and flowering (inflorescences) for an additional five months. The number of second inflorescences on ‘Nellie White’ and ‘Snow Trumpet’ grown from bulbs was also recorded. The results for five plants of each accession are shown in Tables 7 and 8. Results show that L.×formolongo progeny segregate for the reflowering trait.

TABLE 7

| | | | | Days to | |
|---|---|---|---|---|---|
| | | Photo- | Reflowering % (or | Visible | Days to |
| Accession | | period | with buds) | Bud* | reflowering* |
| ‘Nellie White’ | Bulbs | SD^{a} | 0¹ | | |
| | | LD^{a} | 0¹ | | |
| ‘Snow Trumpet’ | Seeds | SD | 0² | | |
| | | LD | 0³ | | |
| L. formosanum | Bulbs | SD | 0¹ | | |
| | | LD | 0¹ | | |
| L. formosanum | Seeds | SD | 0¹ | | |
| | | LD | 20⁴ | 177 | — |
| ‘Raizan No. 1’ | Seeds | SD | 80 (20% resetted) | 140 | 120 |
| (combined seed lots) | | LD | 100 | 110 | 114 |
| ‘Raizan No. 1’ | Seeds | SD | 60 (40% rosetted) | 159 | — |
| (combined seed lots) | | LD | 80 (20% rosetted) | 126 | 110 |
| ‘Sakigake Raizan’ | Seeds | SD | 60 (20% dead, 20% | — | — |
| (combined seed lots) | | | rosetted) | | |
| | | LD | 50 (50% dead) | — | — |
| 00L-13 | Seeds | SD | 20 (80% rosetted) | 190 | — |
| | | LD | 25 (75% rosetted) | 64 | 64 |
| 00L-74 | Seeds | SD | 20 (80% rosetted) | 149 | — |
| | | LD | 40 (60% rosetted) | 110 | 149 |
| 00L-96 | Seeds | SD | 0 (100% rosetted) | | |
| | | LD | 0 (100% rosetted) | | |
| 00L-116 | Seeds | SD | 0 (100% rosetted) | | |
| | | LD | 20 (80% rosetted) | 139 | 145 |
| 00L-136 | Seeds | SD | 0 (100% rosetted) | | |
| | | LD | 20 (80% rosetted) | 167 | 176 |

*Determined as the number of days from visible bud date or flowering of the first shoot of each plant, respectively;
¹No shoots were formed;
²No shoots formed, 20% were dead;
3 (no shoots 40% dead);
4 (40% dead);
^{a}SD, short day; LD, long day;
Dashes indicate that flower buds were formed but they either aborted or never opened (reached anthesis) before the termination of the experiment.

TABLE 8

| | | | Plant | | |
|---|---|---|---|---|---|
| | | Photo- | height | Infl. length | Flower |
| Accession | | period | (cm) | (cm) | buds/shoot |
| Nellie White | Bulbs | SD^{a} | | | |
| | | LD^{a} | | | |
| ‘Snow Trumpet’ | Seeds | SD | | | |
| | | LD | | | |
| L. formosanum | Bulbs | SD | | | |
| | | LD | | | |
| L. formosanum | Seeds | SD | | | |
| | | LD | 29 | — | 1 |
| ‘Raizan No. 1’ | Seeds | SD | 65 | 6 | 1 |
| | | LD | 64 | 11 | 1 |
| ‘Raizan No. 2’ | Seeds | SD | 59 | 5+ | 1 |
| | | LD | 63 | 13 | 1 |
| ‘Sakigake Raizan’ | Seeds | SD | 62 | 10 | — |
| | | LD | 51.2 | 9 | — |
| 00L-13 | Seeds | SD | 65 | 10+ | 1 |
| | | LD | 43 | 14 | 2 |
| 00L-74 | Seeds | SD | 45 | 10+ | 1 |
| | | LD | 51 | 10 | 1 |
| 00L-96 | Seeds | SD | | | |
| | | LD | | | |
| 00L-116 | Seeds | SD | | | |
| | | LD | 61 | 15 | 1 |
| 00L-136 | Seeds | SD | | | |
| | | LD | 65 | 13 | 3 |

*Determined as the number of days from visible bud date or flowering of the first shoot of each plant, respectively;
¹No shoots were formed;
²No shoots formed, 20% were dead;
3 (no shoots 40% dead);
4 (40% dead);
5 (20% rosetted);
^{a}SD, short day; LD, long day;
Dashes indicate that flower buds were formed buy they either aborted or never opened (reached anthesis) before the termination of the experiment.

Parental species (bulbs) did not reflower under either photoperiod, indicating an obligate vernalization requirement. None of the parental Lilium longiflorum plants grown from seed reflowered under either photoperiod, indicating an obligate vernalization requirement for the second shoot. About 20% of the Lilium formosanum plants grown from seed had initiated flower buds under a LD photoperiod. These plants would have eventually reflowered. However, it took 177 days (25.3 weeks) to reach visible bud date. See Table 7. Thus, L. formosanum may be the source of gene(s) for the reflowering trait, although this happened only under LD and the second shoots cannot be termed day neutral. Both L. longiflorum and L. formosanum plants or seeds that did not reflower did not send up any new shoots above the soil line. Apparently, there is an obligate cold treatment (vernalization) requirement for reflowering. About 20-40% of the L. longiflorum and L. formosanum plants died under both photoperiods.

L.×formolongo commercial F₁ hybrids had a reflowering percentage of 60% to 80% under short day conditions. The reflowering percentage under long day conditions was about 50% to 100%. See Table 7. All reflowering Fl hybrid plants had one flower/stem. ‘Raizan No. 1’ took 120 days (17.1 weeks) under SD and 114 days (16.3 weeks) under LD to reflower. ‘Raizan No. 2’ took 110 days (15.7 weeks) under LD to flower. The second flower on Raizan No. 2 reflowering plants had not opened when the experiment was terminated.

Individual non-reflowering plants were either dead or rosetted (new shoots visible above the soil line that did not elongate—requiring vernalization). There were about 20-40% dead or rosetted ‘Raizan Herald’ plants under SD and 0-20% dead or rosetted ‘Raizan Herald’ plants under LD conditions. No F₁ hybrid had 100% reflowering under both photoperiods. These data indicate that L.×formolongo F₁ hybrids segregate for reflowering and for a vernalization requirement. Only ‘Raizan No. 1’ plants grown under long day photoperiod did not segregate for reflowering.

L.×formolongo BC₁ hybrids had a reflowering percentage of 0% to 20% under short day conditions. The reflowering percentage under long day conditions was 25% to 40%. Hybrids 00L-13 and 00L-74 had the highest reflowering percentage in both photoperiods. No BC₁ hybrid had 100% reflowering in both photoperiods. Reflowering BC₁ hybrids had from 1-3 flowers on the second shoot. These data indicate that L.×formolongo BC₁ hybrids also segregate for the reflowering trait and a vernalization requirement.

L.×formolongo BC₁ hybrid 00L-13 took 190 days (27.1 weeks) to reach visible bud date under SD and much longer for reflowering. Under LD, it took 64 days (9.1 weeks) to reach both visible bud date and reflowering. See Table 7. BC₁ hybrid 00L-74 took 149 days (21.3 weeks) to reach visible bud date under short day conditions and longer to reflower. Under LD, it took 110 days (15.7 weeks) for hybrid 00L-74 to reach visible bud date and 149 days (21.3 weeks) to reflower. BC₁ hybrid 00L-96 did not segregate for the reflowering trait under either photoperiod.

BC₁ hybrid 00L-116 did not segregated for the reflowering trait under SD. Hybrid 002-116 took 139 days (19.9 weeks) to reach visible bud date and 145 days (20.7 weeks) to reflower under LD. All of the L.×formolongo BC₁ hybrids had a large percentage of plants that rosetted under both SD and under LD conditions. One BC₁ hybrid, 00L-75-23, had a second shoot in which visible bud date and flowering occurred on the same day as the initial shoot. Thus, the number of days to reach visible bud date or reflowering for the second shoot was zero for this particular hybrid. The greatest difference between visible bud dates was 190 days, for hybrid 00L-13.

### Example 5

### Selection of Winter-Hardy Hybrid Lily Plants

Mature L.×formolongo F₁ hybrid plants were planted and grown outdoors in the spring of 2000. The plants were about 10 months old when transplanted. Some of these plants survived the winter of 2000-2001 (45° N latitude, Minnesota, U.S.A.). There were 90 occurrences during the winter of two consecutive diurnal periods in which the minimum ambient air temperature was from about −10° C. to about +5° C. The number of surviving plants was tabulated in the spring of 2001 and the data are shown in Table 9. Winter-hardiness of the plants was not correlated with frost tolerance of the flowers on these plants. ‘Raizan No. 3’ had 0% winter survival. All other hybrids averaged 14% to 80% winter survival. These results show that L.×formolongo plants segregate for winter hardiness.

Table 9

| Variety | Fraction Surviving Plants | % Surviving |
|---|---|---|
| ‘Sakigake Raizan’ Lot 9097 | 12/15 | 80 |
| ‘Augusta F1’ | 2/14 | 14 |
| ‘Raizan No. 1’ Lot 8159 | 3/15 | 20 |
| ‘Raizan No. 2’ Lot 8148 | 3/16 | 19 |
| ‘Raizan No. 3’ | 0/3 | 0 |
| ‘Nellie White’ | 6/17 | 35 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### LIST OF REFERENCES

Ascher & Peloquin, 1968, Amer. J. Bot., 55:1230-1234.

Baranova, 1972, Biological peculiarities of lilies. The Lily Yearbook of the North American Lily Society, 25:7-20.

Beattie & White, 1993, Lilium—Hybrids and species, pp. 423-454. In: The Physiology of flower bulbs, De Hertogh & Le Nard, Eds. Elsevier, Amsterdam.

De Hertogh, 1996, Holland Bulb Forcer's Guide, Alkemade Printing BV, Lisse, The Netherlands.

Dole & Wilkins. 1996, Acta Hort., 414:295-300.

Dole & Wilkins, 1999, Floriculture Principles and Species, Prentice Hall, Upper Saddle River, N.J.

Horst, 1990, Lily, pp. 707-708. In: Westcott's Plant Disease Handbook, 5^{th} edition. Van Nostrand Reinhold, New York.

Lange & Heins, 1988, Hort. Science, 23:749.

Marourky & Woltz, 1977, J. Am. Soc. Hort. Science, 102:799-804.

McRae, 1998, Lilies. Timber Press, Portland, Oreg.

Miller & Kipliner, 1966, Proc. Am. Soc. Hort. Science, 88:646-650.

Okazaki, 1996, Acta Hort., 414:81-92.

Parks Seed® Flowers and Vegetables, 2002 Catalog, Greenwood, S.C.

Roh & Wilkins, 1973, Florists' Review, 153(3960):19-21, 60-63.

Smyth et al., 1989, Pl. Syst. Evol., 163:53-69.

United States Department of Agriculture (2001) Floriculture Crops 2000 Summary, Sp Cr 6-1 (01) a.

Walters & Wilkins, 1967, Proc. Am. Soc. Hort. Science, 90:433-439.

Weiler & Langhans, 1968, Proc. Am. Soc. Hort. Science, 93:623-629.

Weiler & Langhans, 1972, J. Am. Soc. Hort. Science, 97:176-177.

Wilkins, 1973, Minnesota Horticulture, 101:36-38.

Wilkins, 1980, Easter lilies, In: Introduction to floriculture, R. A. Larson, Ed., Academic Press, New York, pp. 327-352.

Wilkins & Roh, 1970, University of Minnesota's Easter Lily Research Report: Paper No. IX., Minnesota State Florists' Bulletin Dec.: 10-12.

What is claimed is:

1. A method of making a lily plant, comprising selecting one or more L.×formolongo plants that has an average reflowering period of from about 0 to about +60 days under long day photoperiod conditions in a greenhouse environment.

2. The method of claim 1, wherein said one or more selected L.×formolongo plants are grown from seeds.

3. The method of claim 1, wherein said one or more selected L.×formolongo plants are grown from bulbs.

4. The method of claim 1, wherein said one or more selected L.×formolongo plants are not vernalized.

5. The method of claim 1, further comprising the step of propagating said one or more L.×formolongo plants.

6. The method of claim 5, wherein said propagating step comprises asexual propagation.

7. The method of claim 1, further comprising selecting one or more said L.×formolongo plants that form flowers under short day photoperiod conditions in a greenhouse environment.

8. The method of claim 1, further comprising selecting one or more said L.×formolongo plants that form 2 to 3 shoots per bulb in a greenhouse environment.

9. The method of claim 1, further comprising selecting one or more said L.×formolongo plants that have a statistically significantly greater number of flowers when grown under short-day conditions in a greenhouse environment relative to the number of flowers when grown under long-day conditions in a greenhouse environment.

10. The method of claim 1, further comprising selecting one or more said L.×formolongo plants that are capable of surviving two consecutive diurnal periods in which the minimum ambient air temperature is from about −10° C. to about +5° C.

11. The method of claim 1, further comprising selecting one or more said L.×formolongo plants that are incapable of surviving two consecutive diurnal periods in which the minimum ambient air temperature is from about −10° C. to about +5° C.

12. The method of claim 1, further comprising selecting one or more said L.×formolongo plants that flower in the absence of vernalization.

13. A method of making one or more lily bulbs, comprising asexually propagating one or more L.×formolongo plants to form bulbs, said one or more plants selected to have an average reflowering period of from about 0 to about +60 days under long day photoperiod conditions in a greenhouse environment.

14. A method of making one or more lily flowers, comprising growing a plurality of L.×formolongo bulbs to form one or more flowering plants, said bulbs selected to have an average reflowering period of from about 0 to about +60 days under long day photoperiod conditions in a greenhouse environment.

15. The method of claim 14, wherein said bulbs are grown under controlled temperature forcing conditions.

16. The method of claim 14, wherein said bulbs are grown under case cooling conditions.

17. A L.×formolongo plant, wherein said plant has an average reflowering period of from about +0 to about +60 days under long day photoperiod conditions in a greenhouse environment.

18. The plant of claim 17, wherein said reflowering period is from about +0 days to about +7 days.

19. The plant of claim 17, wherein said reflowering period is from about +8 days to about +21 days.

20. The plant of claim 17, wherein said reflowering period is from about +22 days to about +60 days.

21. The L.×formolongo plant of claim 17, wherein said plant forms flowers under short day photoperiod conditions in a greenhouse environment.

22. The L.×formolongo plant of claim 17, wherein said plant forms 2 to 3 shoots per bulb in a greenhouse environment.

23. The L.×formolongo plant of claim 17, wherein said plant has a statistically significantly greater number of flowers when grown under short-day conditions in a greenhouse environment relative to the number of flowers when grown under long-day conditions in a greenhouse environment.

24. The L.×formolongo plant of claim 17, wherein said plant is capable of surviving two consecutive diurnal periods in which the minimum ambient air temperature is from about −10° C. to about +5° C.

25. The L.×formolongo plant of claim 17, wherein said plant is incapable of surviving two consecutive diurnal periods in which the minimum ambient air temperature is from about −10° C. to about +5° C.

26. The L.×formolongo plant of claim 17, wherein said plant flowers in the absence of vernalization.
